(19) United States
(12) Patent Application Publication
(10) Pub. No.: US 2015/0374328 A1
(43) Pub. Date: Dec. 31, 2015

(54) SYSTEMS, METHODS AND DEVICES FOR REMOTE FETAL AND MATERNAL HEALTH MONITORING

(71) Applicants: Jacques Ginestet, Los Gatos, CA (US); Jessica Grossman, La Jolla, CA (US); Byron Hourmand, Vista, CA (US); Holmes Chuang, San Diego, CA (US); Albert Fong, San Diego, CA (US)

(72) Inventors: Jacques Ginestet, Los Gatos, CA (US); Jessica Grossman, La Jolla, CA (US); Byron Hourmand, Vista, CA (US); Holmes Chuang, San Diego, CA (US); Albert Fong, San Diego, CA (US)

(21) Appl. No.: 14/630,546

(22) Filed: Feb. 24, 2015

Related U.S. Application Data
(60) Provisional application No. 61/943,830, filed on Feb. 24, 2014.

(51) Int. Cl. A61B 8/02; A61B 5/024; A61B 5/11; A61B 5/145; A61B 5/00; A61B 5/0488; A61B 5/021; A61B 5/0205

(52) U.S. Cl. CPC A61B 8/02; A61B 5/021; A61B 5/02411; A61B 5/02055; A61B 5/14542; A61B 5/4356; A61B 5/0488; A61B 5/0082; A61B 5/0011; A61B 5/11; A61B 5/0022

(57) ABSTRACT

A fetal and maternal monitoring system is provided, including one or more sensing devices, a central control device which receives signals from, and provides power to, the sensing devices, and a gateway device in wireless communication with the central control device for visualization of data received from the central control device and transmission of the received data to a remote location over a network. The one or more sensing devices may include a fetal heart rate monitor (FHR), a strain gauge tocodynamometer (TOCO), maternal heart rate monitor (MHR), blood pressure monitor or other wearable health or fitness sensing device which require only a basic sensor and rely upon the central control device for processing and power. Lighting elements on the sensing devices provide indications of signal detection and strength. The gateway device will also provide instructions to a user for performing tests and virtual physician visits.

### BACKGROUND

1. Field of the Invention

The present invention relates to remote health monitoring, and more particularly to systems, methods and devices for remote fetal and maternal health monitoring.

2. Related Art

Fetal Distress Syndrome is an abnormal condition during gestation or at the time of delivery, marked by altered heart rate or rhythm and leading to compromised blood flow or changes in blood chemistry. Detection of fetal distress syndrome is done in obstetrics by Cardiotocography, the simultaneous measurement of fetal heart rate and uterine contractions. The change in fetal heart rate as a response to uterine contractions is the diagnostic basis of fetal distress syndrome. See, e.g., “Cardiotocography”, van Geijn, H. P., Textbook of Perinatal Medicine, Parthenon Publishing, 1998, Vol. 2, p. 1424-8. In every-day obstetrics practice, physicians routinely prescribe cardiotocograms to detect fetal distress syndrome.

Cardiotocography, or electronic fetal monitoring (EFM), is a common non-invasive diagnostic technique utilized in obstetrics to detect and determine the extent of Fetal Distress Syndrome. Cardiotocography uses the simultaneous measurement of the fetal heart rate (“cardio”) and the uterine contractions (“toco”) to detect any abnormalities.

Current technology is composed of a central unit, which contains a printer, a Doppler fetal monitor (to register the fetal heart rate), and a tocodynamometer (to register uterine contractions). In currently used equipment, the sensors are affixed to the abdomen of the mother and connected to the central unit via connecting cables.

Typically, a conventional tocodynamometer is a strain gauge attached to a belt around the abdomen of the patient. The strain gauge detects the tension on the uterus wall during contractions. Also conventionally, a Doppler ultrasound transducer measures fetal heart rate. The result is a graphical overlay of both measurements, seen either on a screen or on paper. By comparing changes in fetal heart rate to maternal contractions, the healthcare provider assesses the status of the fetus and determines if fetal distress is present.

Currently, obstetric patients requiring EFM are referred to either a hospital or outpatient clinic setting where monitoring takes place under the physical presence of a technician or nurse. While resting in bed, the sensors are placed on the patient and the sensors are connected to a measuring apparatus with cables, thus limiting the patient's mobility. The measuring apparatus displays two simultaneous graphs, one with the fetal heart rate and the other with the uterine contractions (on paper or screen). The practitioner determines the presence and the severity of Fetal Distress Syndrome based on these two graphs. See, e.g., “Interpretation of the Electronic Fetal Heart Rate During Labor”, American Academy of Family Physicians (1999).

Traditional fetal monitoring systems are relatively bulky, expensive and intended to be used in designated centers (e.g., hospitals or physicians' offices). This arrangement raises several issues.

First, there exists a limited accessibility to fetal monitoring. Currently, in the United States, pregnant mothers must commute to either a physician's office or a designated fetal monitoring center and such specialized facilities are often difficult for patients to access. This means that the pregnant mother should take a trip to the hospital for a monitoring session which puts the burden of time and expense both on the mother and accompanying person(s) as well as the healthcare system. Therefore, with traditional systems, monitoring of pregnant mothers who are not categorized as high risk, is limited to a few times during the course of a pregnancy. Even for high risk pregnancies, typical testing is on the order of 2 times every week during the last trimester. This leads potentially to reduced efficacy of monitoring in terms of missing critical incidents. Immobility of the traditional system also means that pregnant mothers in remote areas and/or in underserved areas with limited access to the healthcare system (e.g., in the case of many developing countries) are not being tested at all.

Second, there is limited mobility of the patient during fetal monitoring. Pregnant mothers who undergo fetal monitoring require a minimum of 45 minutes and up to 4 hours for each monitoring session. During this time the patient must remain in a relaxed position (usually recumbent) connected to the recording device. Putting on and adjusting the position of fetal monitoring system sensors takes substantial amount of time (i.e., on the order of 10-20 minutes). Using the traditional wired fetal monitoring system, in case that the patient needs to move during the test (e.g. goes to bathroom or the like) the setup needs to be removed and placed back afterwards. This adds additional time and cost burden in the hospitals.

Third, there is a lack of remote accessibility to data for evaluation. Currently most cardiotographic devices do not have the capability of digital storage and transfer. The usual manner in which a fetal monitoring study occurs involves a paper tracing that is carried to the health care provider or physician for interpretation, and then stored in the patient's medical record. Often the length of these strips exceeds the capacity for storage for clinical, private physician practices and even hospital systems. Additionally, the lack of digital data transferability means that interpretation of the data is possible only in places where obstetrical specialists are accessible.

Doppler ultrasound is a non-invasive monitoring approach to extract information about moving structures inside the body. It can be used for diagnosis of many cardiovascular conditions as well as in fetal health monitoring. Current ultrasonic technologies rely on bedside monitoring that is limited to the hospital and clinical settings. A major obstacle in transforming the traditional ultrasonic technologies into the emerging wireless health solutions is the significantly high computational complexity of the algorithms that process the plethora of the Doppler shifted data acquired from ultrasound transducers.

With the growing interest in wireless health technologies and their potential applications, efficient design and development of wearable medical devices is becoming unprecedentedly important to researchers in both academia and industry. See, e.g., R. Jafari, S. Ghiasi, and M. Sarrafzadeh, “Medical Embedded Systems,” in Embedded System Design: Topics, Techniques and Trends, ser. IFIP Advances in Information and Communication Technology, A. Rettberg, M. Zanella, R. Düner, A. Gerstlauer, and F. Rammig, Eds. Springer Boston, 2007, vol. 231, pp. 441-444. The main driving factors in designing this new generation of the health paradigm include cost, power consumption, and wearablility, with power consumption being the center of many research efforts due to its dramatic influence on other design objectives. See, e.g., C. Park, P. Chou, Y. Bai, R. Matthews, and A. Hibbs, “An Ultra-wearable, Wireless, Low Power ECG Monitoring System,” in Biomedical Circuits and Systems Conference, 2006. BioCAS 2006. IEEE, December 2006, pp. 241-244; P. Zappi, C. Lombriser, T. Stiefineier, E. Farella, D. Roggen, L. Benini, and G. Troster, “Activity Recognition From On-Body Sensors Accuracy-Power Trade-off By Dynamic Sensor Selection,” Lecture Notes in Computer Science, vol. 4913, p. 17, 2008; V. Leonov, P. Fiorini, S. Sedky, T. Toffs, and C. Van Hoof, “Thermoelectric Mems Generators as a Power Supply for a Body Area Network,” vol. 1, June 2005, pp. 291-294; S. Xiao, A. Dhamdhere, V. Sivaraman, and A. Burdett, “Transmission Power Control in Body Area Sensor Networks for Healthcare Monitoring,” IEEE Journal on Selected Areas in Communications, vol. 27, no. 1, pp. 37-48, 2009; and H. Ghasemzadeh and R. Jafari, “A Greedy Buffer Allocation Algorithm for Power-Aware Communication in Body Sensor Networks,” in Proceedings of the eighth IEEE/ACM/IFIP International Conference on Hardware/Software Codesign and System Synthesis, ser. CODES/ISSS '10. New York, N.Y., USA: ACM, 2010, pp. 195-204.

An important angle of low-power design is the development of efficient signal processing and data reduction algorithms that reduce computation load on the processing units, allowing low-power low-cost processors to be embedded with the wearable device. While much work has been done on designing signal processing algorithms for a variety of sensing modalities such as motion sensors (H. Ghasemzadeh, V. Loseu, and R. Jafari, “Structural Action Recognition in Body Sensor Networks: Distributed Classification Based on String Matching,” IEEE Transactions on Information Technology in Biomedicine, vol. 14, no. 2, pp. 425-435, 2010; A. Barth, M. Hanson, H. Powell, and J. Lach, “Tempo 3.1: A Body Area Sensor Network Platform for Continuous Movement Assessment,” in Wearable and Implantable Body Sensor Networks, 2009. BSN 2009. Sixth International Workshop on, 2009, pp. 71-76.), Electrocardiography (D. Jun, X. Miao, Z. Hong-hai, and L. Wei-feng, “Wearable ECG Recognition and Monitor,” in Computer-Based Medical Systems, 2005. Proceedings. 18th IEEE Symposium on, June 2005, pp. 413-418; M. Ayat, K. Assaleh, and H. Al-Nashash, “Prototype of a Standalone Fetal ECG Monitor,” in Industrial Electronics Applications (ISIEA), 2010 IEEE Symposium on, 2010, pp. 617-622), and photo-plethysmogram sensors (J. Espina, T. Falck, J. Muehlsteff, and X. Aubert, “Wireless Body Sensor Network for Continuous Cuff-less Blood Pressure Monitoring,” in Medical Devices and Biosensors, 2006. 3rd IEEE/EMBS International Summer School on, 2006, pp. 11-15), ultrasonic signal processing for stringent constrained computing platforms has not been studied in the past.

Traditional ultrasound technologies have been used in a variety of application domains such as ultrasound imaging (E. J. Gussenhoven, C. E. Essed, C. T. Lancée, F. Mastik, P. Frietman, F. C. van Egmond, J. Reiber, H. Bosch, H. van Urk, J. Roelandt, and N. Bom, “Arterial Wall Characteristics Determined by Intravascular Ultrasound Imaging: An in vitro Study,” Journal of the American College of Cardiology, vol. 14, no. 4, pp. 947-952, 1989, ACC Anniversary Seminar) to produce pictures of the inside of the body, blood flow monitoring (A. Azhim, J. Yamaguchi, Y. Hirao, Y. Kinouchi, H. Yamaguchi, K. Yoshizaki, S. Ito, and M. Nomura, “Monitoring Carotid Blood Flow and ECG for Cardiovascular Disease in Elder Subjects,” in Engineering in Medicine and Biology Society, 2005. IEEE-EMBS 2005. 27th Annual International Conference of the, 2005, pp. 5495-5498) to measure velocity of blood flow in different arteries for use in monitoring cardiovascular diseases, and Cardiotocography (C.-Y. Chen, J.-C. Chen, C. Yu, and C.-W. Lin, “A Comparative Study of a New Cardiotocography Analysis Program,” in Engineering in Medicine and Biology Society, 2009. EMBC 2009. Annual International Conference of the IEEE, September 2009, pp. 2567-2570) to measure fetal heart rate and assess the effect of uterine contractions on fetal heart rate. However, the main challenge in transition from traditional ultrasound technologies to wearable platforms is the demand for a very high computational power. Compared to the other sensing modalities, ultrasound signals require a relatively high sampling frequency, producing large volumes of data that need to be processed. For instance, in a blood flow monitoring application, relevant information may appear in the frequency band of 100-4200 Hz, which may require a sampling frequency of 10 kHz as used in Azhim, et al, above. Moreover, a minimum sampling rate of 1600 Hz for capturing fetal movements is suggested in C.-Y. Chen, J.-C. Chen, C. Yu, and C.-W. Lin, “A Comparative Study of a New Cardiotocography Analysis Program,” in Engineering in Medicine and Biology Society, 2009. EMBC 2009. Annual International Conference of the IEEE, September 2009, pp. 2567-2570. The large volume of sampled ultrasonic signals needs to undergo fast signal conditioning algorithms in order to extract relevant information in real-time.

As to patents, Rapoport, U.S. Pat. No. 5,257,627, discloses a portable apparatus for the non-invasive, simultaneous, self-testing of fetal and maternal signals. It includes a user display to indicate that the device is operational, an ultrasonic system to detect fetal heart rate connected to said device, a detection system for maternal input signal connected to said device, wherein the device has signal processor for simultaneously processing fetal heart rate and maternal input signals, and also has a communication linking means for the simultaneous transmission of fetal heart rate and maternal input data to a remote output device.

Lewis et al., U.S. Pat. No. 6,115,624, discloses an intrauterine catheter device for monitoring fetal and/or maternal heart rate, including an elongate housing having proximal and distal portions, an array of ECG electrodes on the distal portion and one or more acoustic or other mechanical sensors on the distal portion. A pressure transducer may also be provided on the distal portion. Processor circuitry compares the ECG signal with the output signal of the acoustic sensor to derive fetal and/or maternal heart rate. An intrauterine catheter device is also provided, including a reference electrode on its distal portion, and an array of active electrodes spaced apart from one another on the distal portion. The device may also include a pressure transducer on the distal portion and processor circuitry coupled to the array of active electrodes and/or to the reference electrode for deriving fetal ECG from signals produced by the array of active electrodes. Alternatively, the array of electrodes and acoustic sensors may be provided on a flexible pad that may be secured to the abdomen of a pregnant mother. An intrauterine catheter device is also provided, including a plurality of lumens communicating with a differential pressure transducer provided on its distal portion, and having a zeroing switch on its proximal portion for resetting the pressure transducer in situ.

Powell et al., U.S. Patent Application No. 2006/0149597, makes the following statements in the patent. It is said to provide a data processing tool for the viewing of real-time, critical patient data on remote and/or mobile devices. It is said that the tool renders graphical data on the screen of the remote device in a manner that makes it practical for the health care provider to accurately and timely review the data for the purpose of making an informed decision about the condition of the patient. Charting control is established and implemented using the latest GDI+, GAPI and PDA drawing techniques. The charting components provide landscape support, an ability to overlay patient data and patient images, zoom in/zoom out, custom variable speed scrolling, split screen support, and formatting control. It is said that the methodology operates as an asynchronous application, without sacrificing processing time in the mobile/handheld device. The methodology allows the critical patient data to be streamed in real-time to the handheld device while conserving enough CPU power to simultaneously allow the end user to interact at will with the responsive display application. The methodology is structured using object oriented concepts and design patterns. Each logical tier of the methodology, from the data access objects and the charting control objects, to the user interface objects, is structured with precise interfaces. The methodology implements an IT management console that allows system managers to monitor the exchange of data between hospital systems and the primary database, including all patient data packets, notifications and alerts, connected remote devices.

Hayes-Gill et al., U.S. Pat. No. 7,532,923, discloses an apparatus for detecting the heart rate of a fetus. The apparatus includes at least two detectors for detecting heart beats of the fetus, each detector comprising at least two electrodes for detecting ECG signals. A processor, which is coupled to the detectors, is used to process the ECG signals received from each detector and determine the heart rate of the fetus.

James et al., U.S. Patent Application No. 2007/0213672 discloses a monitor for fetal behavior by receiving ECG data from a set of electrodes attached to a material body. A waveform pre-processor identifies a succession of fetal ECG complex waveforms within the received data and a waveform processor determines differences in the processor succession of fetal ECG complex waveforms over time. An event logger determines from the determined differences a number of fetal movements during the period of time. Fetal spatial presentation and/or position within the uterus may also be determined from fetal ECG data acquired from a plurality of electrodes positioned on the maternal abdomen in a predetermined configuration. A number of fetal ECG complex waveforms are identified within the data, and each of the waveforms is compared with a set of predetermined fetal ECG complex templates ascribed to the predetermined electrode configuration to determine a template that best matches the identified fetal ECG waveforms.

Hayes-Gill et al., WO 2001/004147, discloses a system for detecting uterine activity which uses cutaneous electrodes on the maternal abdomen to obtain electrophysiological signals that can be used to obtain fetal and maternal heart rate. The apparatus includes a first input for receiving electrical signals from the cutaneous electrodes and a second input for receiving movement signals indicative of a movement of the maternal body from a movement detector. A signal processor separates a uterine electromyogram signal from fetal and maternal heart rate signals and filters out motion artifacts from the electromyogram using the movement signals. An output presents electrohysterogram (EHG) data from the uterine electromyogram signal.

Against this background is a compelling need to both bring healthcare to the underserved population, as well as to deliver more efficacious and cost effective healthcare. Further, there is a need to provide a marriage of wireless technologies in a way that is both safe and effective. Despite these compelling needs, the difficulty in detecting Fetal Distress Syndrome remains.

Therefore, what is needed is a system and method that overcomes these significant problems found in the conventional systems as described above.

### SUMMARY

A fetal and maternal health monitoring system is provided, including one or more sensing devices, a central control device in powered communication with the one or more sensing devices, and a wireless gateway device in wireless communication with the central control device for visualization of data received from the central control device and transmission of the received data to a remote location over a network. The one or more sensing devices may include a fetal heart rate monitor (FHR), a strain gauge tocodynamometer (TOCO), maternal heart rate monitor (MHR), blood pressure monitor or other wearable health or fitness sensor which relies on the central control device for processing and power. Each sensing device therefore requires only a basic sensor, as the power and signal processing is provided by the central control device. Lighting elements on the sensing devices provide indications of signal detection and strength. The gateway device will also provide instructions to a user for performing tests and virtual physician visits.

Other features and advantages of the present invention will become more readily apparent to those of ordinary skill in the art after reviewing the following detailed description and accompanying drawings.

### BRIEF DESCRIPTION OF THE DRAWINGS

The structure and operation of the present invention will be understood from a review of the following detailed description and the accompanying drawings in which like reference numerals refer to like parts and in which:

FIG. 1 is an illustration of exemplary devices for wireless fetal and maternal monitoring, according to an embodiment of the invention;

FIG. 2A is an illustration of a sensing device and a central control device for remote monitoring of fetal heart rate, uterine contractions and maternal heart rate, according to an embodiment of the invention;

FIG. 2B is an illustration of a gateway device for displaying and transmitting data collected by the sensing device and central control device, according to an embodiment of the invention;

FIGS. 3A-3F are graphical user interface illustrations of instructions presented to a user during a remote physician visit, according to one embodiment of the invention;

FIG. 4A is a block diagram illustrating an exemplary system for remote wireless fetal and maternal monitoring according to an embodiment of the invention;

FIG. 4B is an illustration of a system for facilitating a remote physician visit, according to one embodiment of the invention;

FIG. 5 is a flow diagram illustrating an exemplary method for wireless fetal and maternal monitoring, according to an embodiment of the invention;

FIG. 6 is a flow diagram illustrating an exemplary method for remote interaction and communication between a clinician and a mother utilizing the exemplary system for wireless fetal and maternal monitoring, according to an embodiment of the invention; and

FIG. 7 is a block diagram illustrating an example wired or wireless processor enabled system that may be used in connection with various embodiments described herein.

### DETAILED DESCRIPTION

Certain embodiments disclosed herein provide for systems and methods for remote fetal and maternal monitoring using wearable sensing devices which are provided with power and signal processing through connections with a central control unit. The central control unit wirelessly transmits the signal data to a gateway device which can then analyze, display and further transmit the data to a remote location for analysis. The gateway device is configured with a display and other interactive functionality to display instructions to a user for performing tests with the sensing devices or interacting with a remote user during a virtual office visit. The remote fetal and maternal monitoring systems and methods therefore provide for frequent, convenient remote monitoring of maternal and fetal health.

The central control unit is configured to provide proprietary connections with a plurality of different sensing devices in order to provide power to the sensing devices and receive the raw signal data, eliminating the need for each sensing device to have an onboard power source, processor or wireless means of communication. This configuration provides the most cost-effective design for a sensing device. The central control unit is responsible for processing the received signals and wirelessly transmitting the processed signal data to the gateway device for analysis and comparison with signal data from other sensing devices, display to the user and transmission to a remote location, such as a physician or clinician device.

The sensing devices may be configured with only a basic sensor and be disposable, for example for temporary use by an expectant mother during pregnancy. The sensing devices may also be configured with visual indicators that guide a patient when positioning the sensing device to detect a desired signal. The visual indicators may be LED lights and provide various colors or illumination patterns that correspond to the reception of a desired signal and the strength of that signal.

The gateway device may be a portable electronic device with a display and user interface input devices that allow the user to see the collected data and view any analysis of the data as it relates to potential health problems, diagnoses or messages from a physician or clinician in communication with the user. The gateway device may provide instructions for positioning the sensing devices or real-time feedback on the signals being received by the sensing devices to help the user better position the sensing devices or remain still when a particular sensing device is active. The gateway device may also provide instructions on the steps for performing specific tests, such as the use of a fetal heart rate (FHR) monitor and a tocodynamometer (TOCO) to collect data on the FHR and maternal uterine contractions for diagnosing fetal distress syndrome.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

### I. Sensing Devices

The sensing devices are configured to be worn by a user in order to measure various aspects of the user's health, such as heart rate, blood pressure, temperature, oxygen levels, movement, sleep, activity and exercise. For an expectant mother, sensing devices which measure fetal activity and vital signs may also be utilized, such as the FHR monitor and TOCO described above. Each sensing device may be configured with a unique sensor which detects a particular type of signal from the user and transmits it to the central control device. The FHR, for example, may use a piezoelectric transducer, while other devices may use ultrasound transducers, light reflection or electrodes. The signals may be directly transmitted to the central control device without requiring any processing or filtering of the signals at the sensing device, eliminating the need for additional components within the sensing device. Therefore, the sensing device may be configured without a power source, processor or analog filtering components in order to minimize the manufacturing cost of the sensing device and allow it to be disposed of after a short period of use.

In one embodiment, the sensing devices may be connected to the central control device with a proprietary connector. The connector may be a cable with wires for providing power to the sensing device from the central control unit and transmitting one or more signals from the sensing device to the central control unit. For example, a maternal heart rate (MHR) monitor uses two wires, while a TOCO uses three wires, and an FHR uses twelve. Each sensing device may also use a different type of wire for powering the sensing device depending on the amount and type of power needed.

In one embodiment, a cardiotocography sensing system includes a fetal heart rate (FHR) monitoring unit and a maternal uterine contraction monitoring unit to provide FHR and contraction information of a mother and fetus. The FHR monitoring device may be a Doppler ultrasound device which must be carefully positioned over the abdomen area to pinpoint the location of the fetus' heart, although the FHR may utilize a steerable ultrasound device to minimize the difficulties of positioning the FHR. The uterine contraction monitoring device may be a contraction actuator actuatable upon a maternal uterine contraction, which optionally is an EMG sensor. In one embodiment, the uterine contraction monitoring device is a tocodynamometer (TOCO).

The sensing devices may also include one or more visual indicators on a housing of the sensing device to indicate whether the device is receiving power, whether the device is ready to receive a signal, whether the device is detecting a signal and the strength of the signal. By positioning the visual indicators on the sensing device itself, the user is able to easily view the visual indicator while positioning the sensing device on the body instead of looking at a visual indicator on the central control device or gateway device while also attempting to simultaneously look at the sensing device in a separate location. Additional visual indicators may provide indications of communication with the central control device or gateway device. In one embodiment, the visual indicators may be a series of LEDs embedded within the housing which are capable of displaying a variety of colors or flashing patterns to provide specific indications of the status of the device. One LED may be configured to aid the user in positioning the device to obtain a strong signal, for example by changing color as a signal gets stronger or displaying a flashing pattern that changes to a solid light once a strong signal is acquired. The particular strength of the signal needed to change the colors and/or flashing patterns may be controlled by the central control unit and may be customized for each type of sensing device based on the type of signals being acquired. The central control device may also provide the necessary signal processing algorithms for analyzing the signals to determine the corresponding color and flashing patterns on the sensing devices.

In one example, LEDs corresponding to the FHR and TOCO may display a continuous light of a certain color when the respective unit is ready, and display a flashing light of the same color when the unit is sensing data. A different color may be used to indicate the strength or quality of the signal from each of the units, which may provide an indication to the mother that the sensing device needs to be re-positioned. A green color may indicate a strong signal, while a yellow color indicates a weak signal and a red color indicates no detectable signal. A third LED may be configured to display a continuous color when the sensing device is ready to communicate with the central control device and a flashing pattern of the same color when communication is occurring. If communication fails, a different color may appear on the third LED.

### II. Central Control Device

The central control device communicates with attached sensing devices to receive signals detected by the sensing devices, process the signals and forward the data to a desired destination, such as the gateway device. The central control device also powers any connected sensing devices to avoid the need for individual power supplies in each sensing device. By providing the processing and power for connected peripherals, the cost for each peripheral sensing device is minimized. Furthermore, the central control device may be configured to communicate wirelessly with the gateway device and communicate the data from all of the various sensing devices over a single communication protocol, such as WiFi® or Bluetooth®. In one embodiment, the central control device operates using standardized communication protocols which allow the central control device to communicate with other wireless health devices, such as a fitness or activity tracking device or a continuous glucose monitor. The central control device may be a wireless wearable device similar to the sensing device, and may be worn on the patient's body, such as around a wrist or neck.

The central control device may be implemented with proprietary connection ports to receive the connector for any type of sensing device. The connector, such as a cable, will provide for an electrical connection between the central control device and the sensing device, as well as communication between the two. In one embodiment, the communication may be accomplished over the electrical connection to reduce the number of wires running between the two devices. However, as noted above, many sensing devices have unique signals and data transmission protocols that require a specific number of wires and connectors. Therefore, the central control device acts as a central hub for the sensing devices. In one embodiment, each connector is uniquely shaped to avoid confusion when attaching the sensing devices to the central control device, and each connector may further be color-coded to match the corresponding connection port on the central control device.

The central control device incorporates at least one processor and memory configured to receive and process the signals from each of the sensing devices, communicate with the sensing devices to control the detection of signals and forward data from the processed signals to the gateway device. In one embodiment, the central control unit is configured with a digital signal processing (DSP) chip for signal processing and a very low power processor for communicating with the gateway unit.

A plurality of visual indicators may also be configured on the central control device to indicate whether the central control device is powered on, the status of an internal rechargeable battery, the status of the central control device, whether it is communicating with one or more of the sensing devices, whether it is communicating with the gateway device, and whether it is in communication with the gateway device. In one embodiment, the central control device is configured with a heart rate monitor designed to contact a skin surface of the user while the central control device is being worn so that the heart rate can also be measured. The central control device may be programmed to activate the sensing devices and initiate communication with the gateway device if the heart rate monitor begins detecting a signal in order to provide an automatic initiation of the sensing devices. The central control device may then be configured to provide sufficient contact with the skin surface for the heart rate monitor or pulse oximeter to provide an accurate measurement of blood flow using reflectance pulse oximetry, for example.

### III. Gateway Device

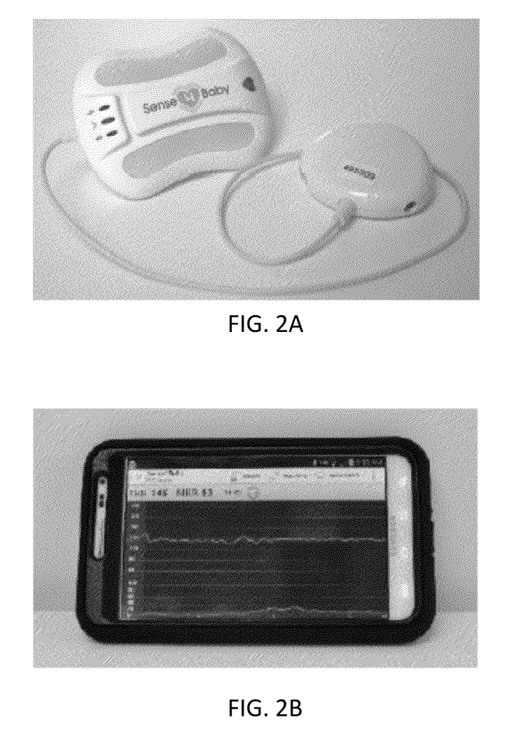

As illustrated in FIG. 2B, the gateway device may be a portable electronic device configured to display the processed signal data received from the central control unit and transmit the signal data to a remote server for analysis, storage or remote monitoring (as described below). The gateway device also functions as a computing device with a processor running an application programmed to receive the signal data, analyze the signal data and provide visual representations of the signals on a display screen. The gateway device may also provide a user interface with menus for organizing and viewing the different signal data and identifying abnormalities in the signal data based on programmed ranges or thresholds of the signals.

The gateway device also communicates with other remote devices, such as a remote server, desktop computing device or other portable electronic device to display the visual representations or provide summaries of the data for analysis by a remote user such as a physician or clinician at a remote location. The remote user may communicate with the gateway device to provide a diagnosis, indicate the need for further testing or an in-person visit by the user of the sensing devices.

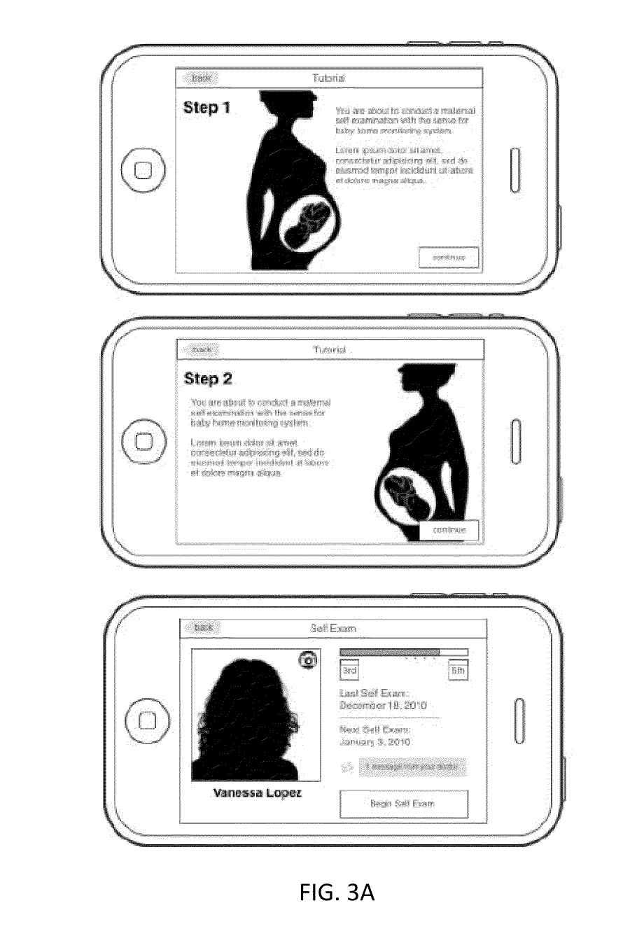

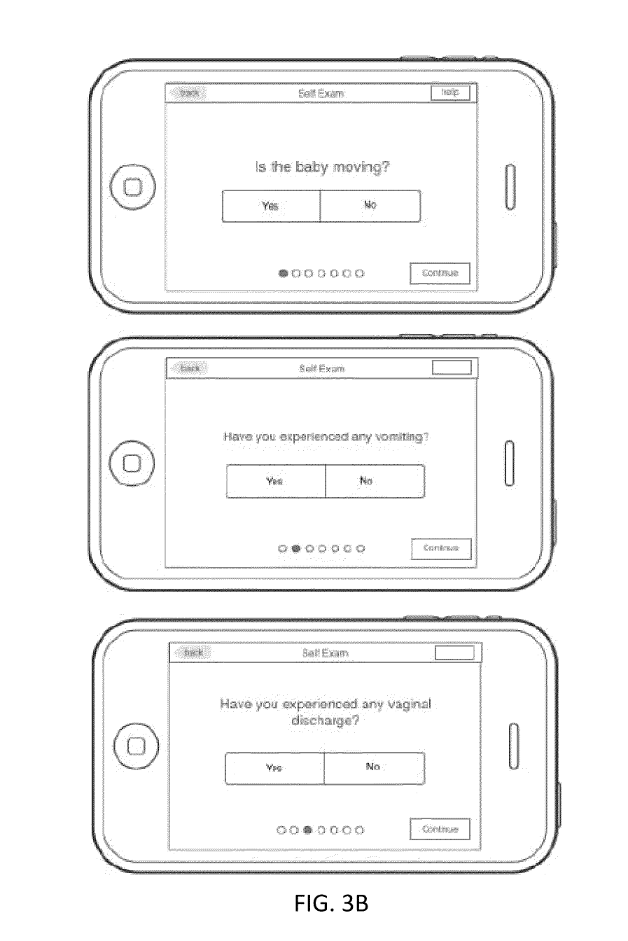

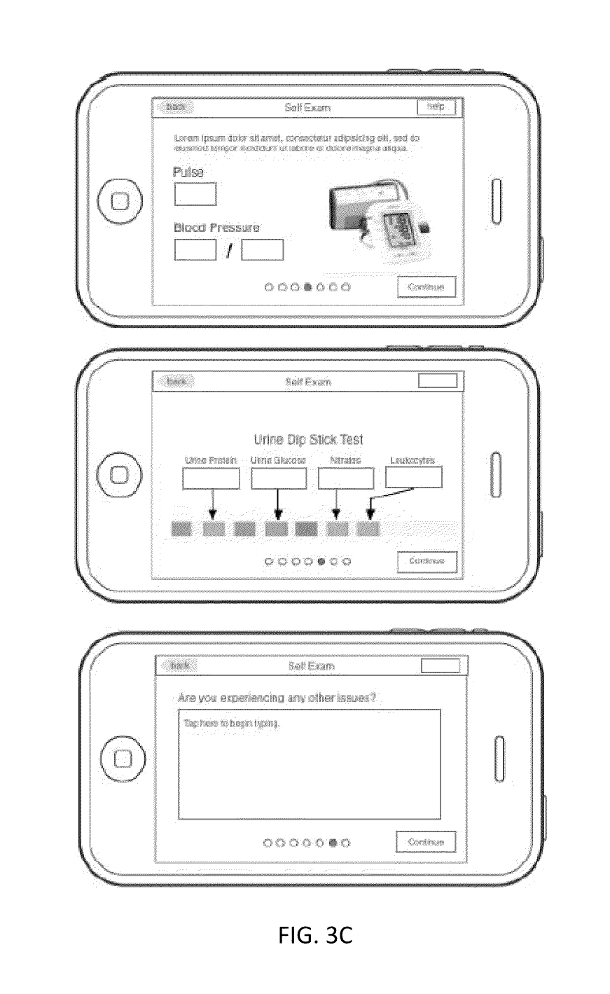

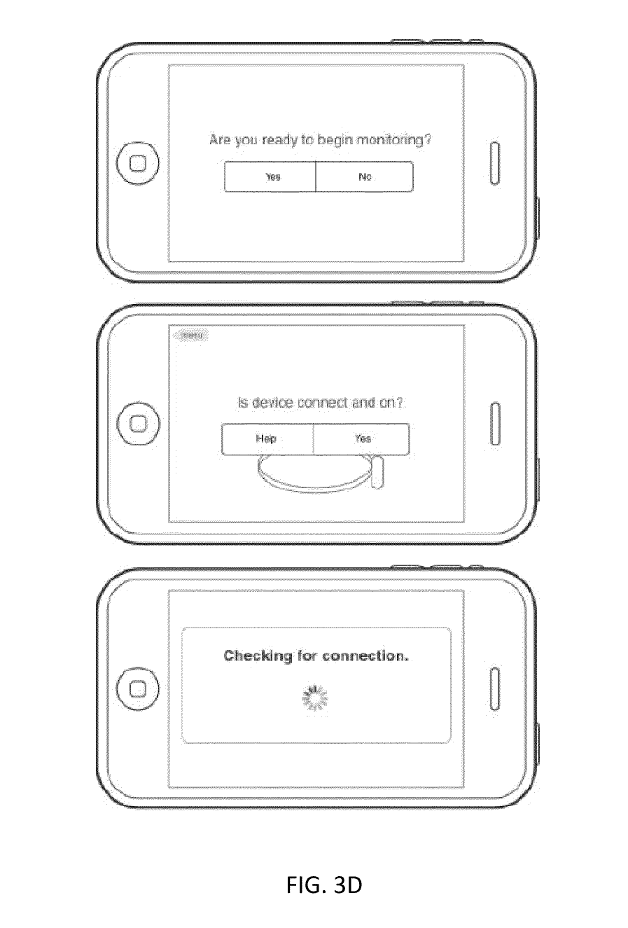

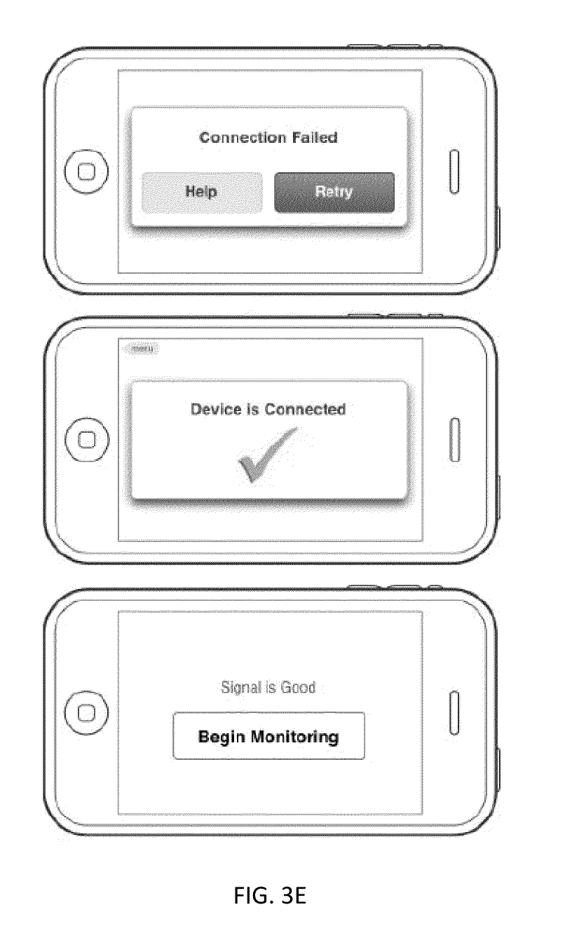

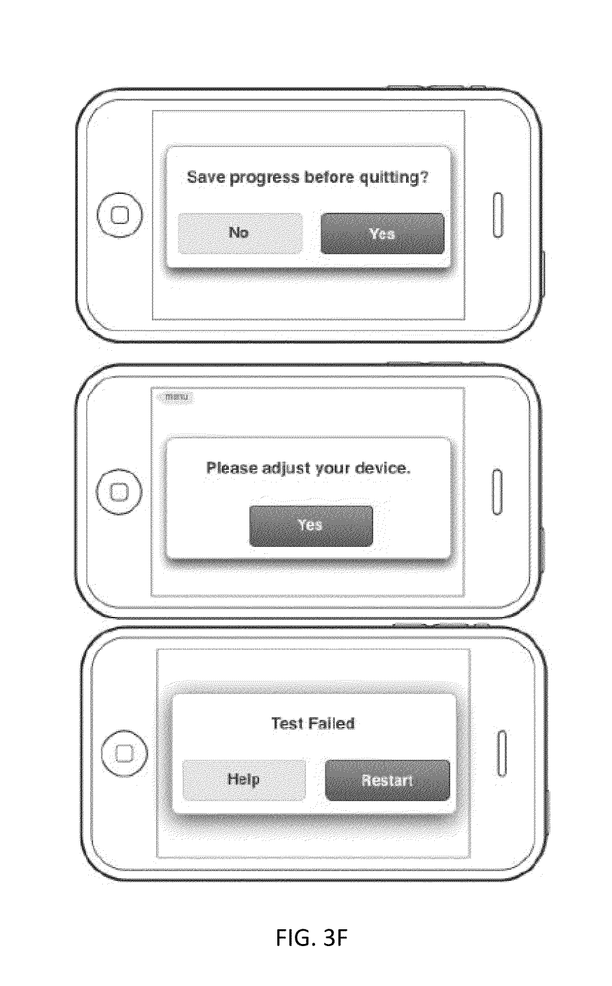

In one embodiment illustrated in FIGS. 3A-3F, the gateway device may provide the user with a set of instructions via a graphical user interface (GUI) that will explain a process for performing one or more tests with the sensing devices. The GUI may require interaction with the user via input devices or a touchscreen display of the gateway device in order to communicate with the central control device to perform tests with the sensing devices. The gateway device may therefore provide the user with a virtual doctor appointment through the performance of tests, display of results and analysis by a physician or clinician. The physician or clinician may be available in real time to review the test results and discuss the results with the user via a messaging interface, a voice communication interface or even a video communication interface.

In one embodiment, the gateway device may provide location-based services using a GPS unit or other positioning software to correlate the user's test results with their location. Abnormal test results may be correlated with locations to determine if certain locations or environments are causing the abnormal results.

In one embodiment, the gateway device may also be configured with an image capture device for capturing an image of glucose urine test strip, which may be analyzed using image processing software to determine a glucose level. The image capture device may also be used to capture images of the skin to diagnose dermatological conditions or other disease symptoms, and generally any other image of the user that may be useful for diagnosis.

### IV. Monitoring Applications

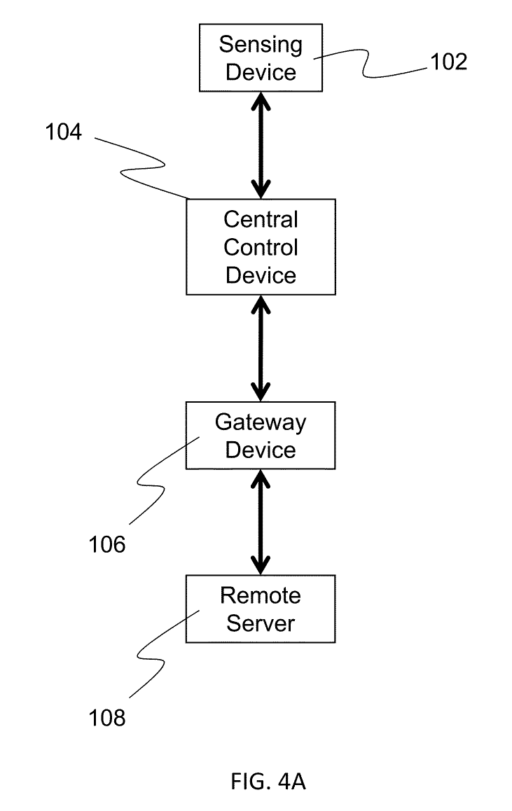

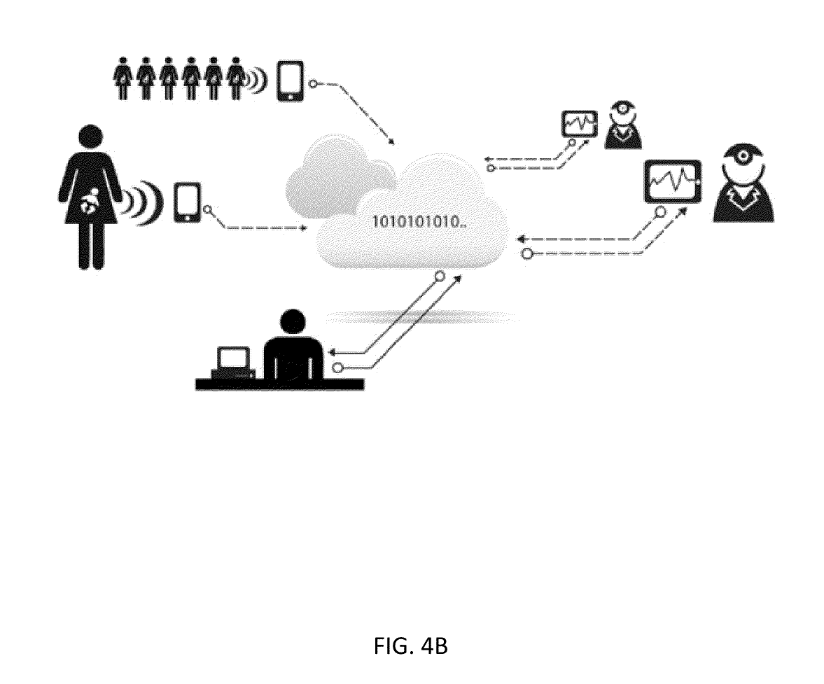

As illustrated in FIG. 4A, the sensing devices, central control device and gateway device provide an overall monitoring system for monitoring the vital signs of a fetus and mother, with specific regard to the FHR, MHR and uterine contractions. The overall monitoring system is illustrated in FIG. 4B, where data from numerous patients are transmitted from the gateway devices over a network to physicians, who analyze the data and provide feedback to the patients. As illustrated herein, an administrator may also be connected with the network to manage the transmission and security of the data.

In one embodiment, the system provides for at least one wearable sensing device which includes a fetal heart rate (FHR) monitor and a tocodynamometer (TOCO) to collect data on the FHR and maternal uterine contractions. A wearable central control device is in wired communication with the sensing device to provide power to the sensing device, manage and control the sensing device and receive the collected data. The central control device may also incorporate a pulse oximeter to determine the maternal heart rate (MHR) at the location where the central control device is worn, such as a wrist. The data collected by the sensing device and central control device may then be wirelessly transmitted to a gateway device, such as a smartphone, which visualizes the data for the mother and transmits the data to a remote server for software-based analysis or review by a clinician. The clinician or software may then communicate with the mother via the gateway device to indicate the health of the mother and baby, provide medical advice or request that the mother schedule a follow up visit to further analyze identified issues. The gateway device may also utilize a built-in image capture device to capture an image of a urine test strip which can be analyzed via image processing software to determine an amount of protein, urea, leukocytes, ketones, etc. in the urine. Additional gateway device hardware, such as location-based antennas, accelerometers, gyroscopes and other sensors, may be used to correlate the collected FHR, contraction and MHR data with the mother's location, environment and activities.

### V. Monitoring Methods

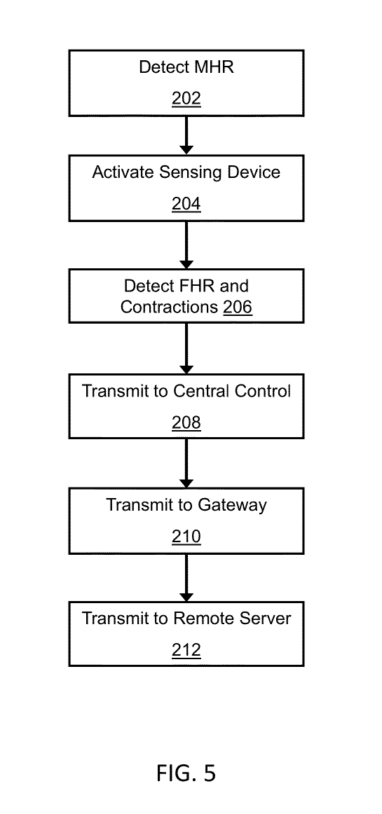

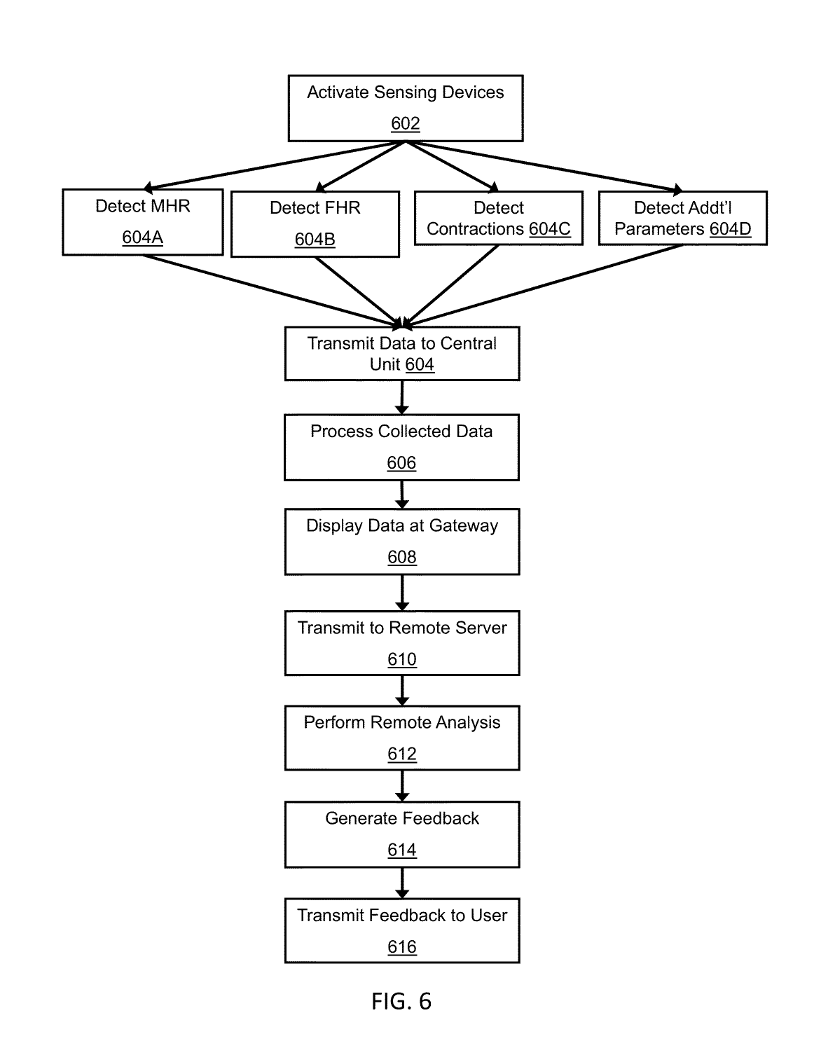

FIGS. 5 and 6 illustrate various methods for fetal and maternal monitoring. FIG. 5 illustrates a flowchart of the steps for performing a cardiotocographic test by measuring the MHR, FHR and uterine contractions with sensing devices, then transmitting the signals first to the central control device for processing, then to the gateway device for display and analysis, and finally to a remote server for analysis and review by a remote user. In FIG. 6, a method of interaction between a user and a remote physician is illustrated, whereby the collected data is displayed to the user at the gateway device, then transmitted to a remote server for third party analysis, after which feedback is generated and transmitted back to the user on the gateway device.

### VI. Computer Embodiment

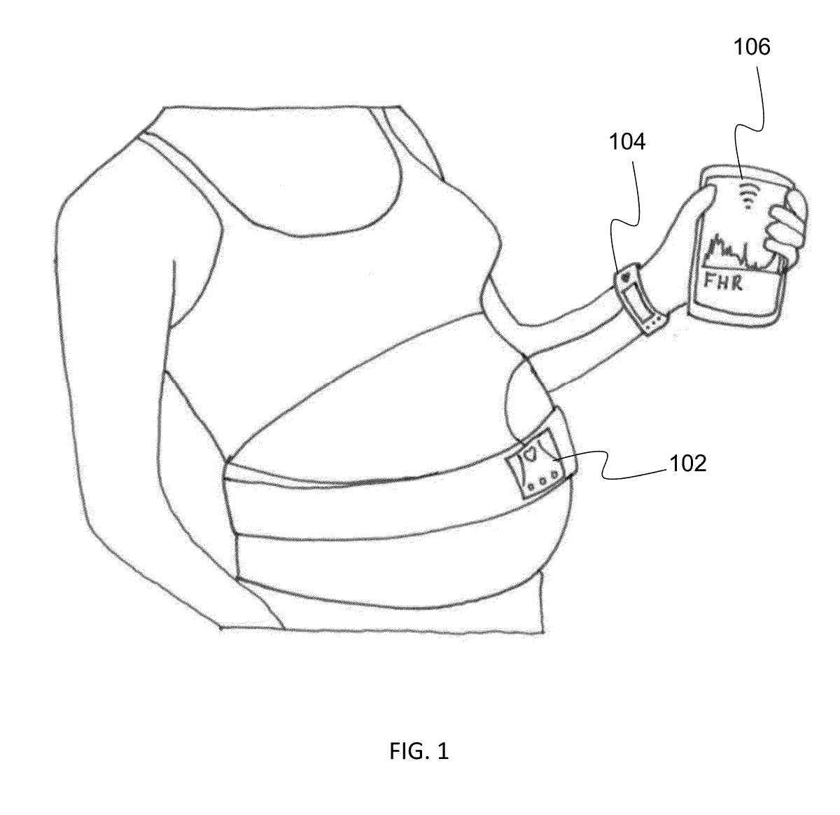

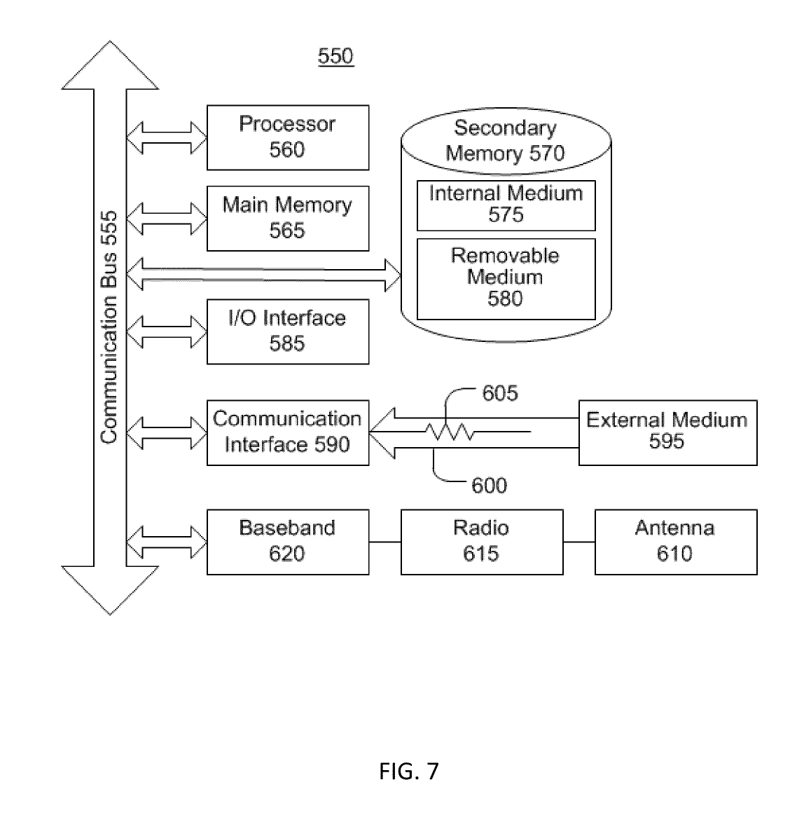

FIG. 7 is a block diagram illustrating an example wired or wireless system 550 that may be used in connection with various embodiments described herein. For example the system 550 may be used as or in conjunction with the fetal and maternal monitoring system, as previously described with respect to FIGS. 1-6. The system 550 can be a conventional personal computer, computer server, personal digital assistant, smart phone, tablet computer, or any other processor enabled device that is capable of wired or wireless data communication. More particularly, the system 550 may represent the central control device, gateway device or remote server. Other computer systems and/or architectures may be also used, as will be clear to those skilled in the art.

The system 550 preferably includes one or more processors, such as processor 560. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 560.

In one embodiment, the central control device is configured with more than one processor in order to separately process the incoming signals from a plurality of sensing devices, while a yet further processor is responsible for handling communication with the gateway device. In another embodiment, the gateway device may also be configured with a plurality of processors, with one processor configured to receive and process data from the central control device, a second processor configured to generate graphical user interfaces to display the received data to the user on a display of the gateway device and a third processor to communicate with the remote server. For a portable electronic device such as the central control device and gateway device, the processors may be low power processors to reduce power consumption on the devices' batteries.

The processor 560 is preferably connected to a communication bus 555. The communication bus 555 may include a data channel for facilitating information transfer between storage and other peripheral components of the system 550. The communication bus 555 further may provide a set of signals used for communication with the processor 560, including a data bus, address bus, and control bus (not shown). The communication bus 555 may comprise any standard or non-standard bus architecture such as, for example, bus architectures compliant with industry standard architecture (“ISA”), extended industry standard architecture (“EISA”), Micro Channel Architecture (“MCA”), peripheral component interconnect (“PCI”) local bus, or standards promulgated by the Institute of Electrical and Electronics Engineers (“IEEE”) including IEEE 488 general-purpose interface bus (“GPIB”), IEEE 696/S-100, and the like. These standards may be applicable to the remote server, while additional or varying standards may apply to portable electronic devices such as the central control device or sensing devices.

System 550 preferably includes a main memory 565 and may also include a secondary memory 570. The main memory 565 provides storage of instructions and data for programs executing on the processor 560. The main memory 565 is typically semiconductor-based memory such as dynamic random access memory (“DRAM”) and/or static random access memory (“SRAM”). Other semiconductor-based memory types include, for example, synchronous dynamic random access memory (“SDRAM”), Rambus dynamic random access memory (“RDRAM”), ferroelectric random access memory (“FRAM”), and the like, including read only memory (“ROM”).

The secondary memory 570 may optionally include a internal memory 575 and/or a removable medium 580, for example a floppy disk drive, a magnetic tape drive, a compact disc (“CD”) drive, a digital versatile disc (“DVD”) drive, etc. The removable medium 580 is read from and/or written to in a well-known manner. Removable storage medium 580 may be, for example, a floppy disk, magnetic tape, CD, DVD, SD card, etc.

The removable storage medium 580 is a non-transitory computer readable medium having stored thereon computer executable code (i.e., software) and/or data. The computer software or data stored on the removable storage medium 580 is read into the system 550 for execution by the processor 560.

In alternative embodiments, secondary memory 570 may include other similar means for allowing computer programs or other data or instructions to be loaded into the system 550. Such means may include, for example, an external storage medium 595 and an interface 570. Examples of external storage medium 595 may include an external hard disk drive or an external optical drive, or and external magneto-optical drive.

Other examples of secondary memory 570 may include semiconductor-based memory such as programmable read-only memory (“PROM”), erasable programmable read-only memory (“EPROM”), electrically erasable read-only memory (“EEPROM”), or flash memory (block oriented memory similar to EEPROM). Also included are any other removable storage media 580 and communication interface 590, which allow software and data to be transferred from an external medium 595 to the system 550.

System 550 may also include an input/output (“I/O”) interface 585. The I/O interface 585 facilitates input from and output to external devices. For example the I/O interface 585 may receive input from a keyboard or mouse and may provide output to a display. The I/O interface 585 is capable of facilitating input from and output to various alternative types of human interface and machine interface devices alike.

System 550 may also include a communication interface 590. The communication interface 590 allows software and data to be transferred between system 550 and external devices (e.g. printers), networks, or information sources. For example, computer software or executable code may be transferred to system 550 from a network server via communication interface 590. Examples of communication interface 590 include a modem, a network interface card (“NIC”), a wireless data card, a communications port, a PCMCIA slot and card, an infrared interface, and an IEEE 1394 fire-wire, just to name a few.

Communication interface 590 preferably implements industry promulgated protocol standards, such as Ethernet IEEE 802 standards, Fiber Channel, digital subscriber line (“DSL”), asynchronous digital subscriber line (“ADSL”), frame relay, asynchronous transfer mode (“ATM”), integrated digital services network (“ISDN”), personal communications services (“PCS”), transmission control protocol/Internet protocol (“TCP/IP”), serial line Internet protocol/point to point protocol (“SLIP/PPP”), and so on, but may also implement customized or non-standard interface protocols as well.

Software and data transferred via communication interface 590 are generally in the form of electrical communication signals 605. These signals 605 are preferably provided to communication interface 590 via a communication channel 600. In one embodiment, the communication channel 600 may be a wired or wireless network, or any variety of other communication links. Communication channel 600 carries signals 605 and can be implemented using a variety of wired or wireless communication means including wire or cable, fiber optics, conventional phone line, cellular phone link, wireless data communication link, radio frequency (“RF”) link, or infrared link, just to name a few.

Computer executable code (i.e., computer programs or software) is stored in the main memory 565 and/or the secondary memory 570. Computer programs can also be received via communication interface 590 and stored in the main memory 565 and/or the secondary memory 570. Such computer programs, when executed, enable the system 550 to perform the various functions of the present invention as previously described.

In this description, the term “computer readable medium” is used to refer to any non-transitory computer readable storage media used to provide computer executable code (e.g., software and computer programs) to the system 550. Examples of these media include main memory 565, secondary memory 570 (including internal memory 575, removable medium 580, and external storage medium 595), and any peripheral device communicatively coupled with communication interface 590 (including a network information server or other network device). These non-transitory computer readable mediums are means for providing executable code, programming instructions, and software to the system 550.

In an embodiment that is implemented using software, the software may be stored on a computer readable medium and loaded into the system 550 by way of removable medium 580, I/O interface 585, or communication interface 590. In such an embodiment, the software is loaded into the system 550 in the form of electrical communication signals 605. The software, when executed by the processor 560, preferably causes the processor 560 to perform the inventive features and functions previously described herein.

The system 550 also includes optional wireless communication components that facilitate wireless communication over a voice and over a data network. The wireless communication components comprise an antenna system 610, a radio system 615 and a baseband system 620. In the system 550, radio frequency (“RF”) signals are transmitted and received over the air by the antenna system 610 under the management of the radio system 615.

In one embodiment, the antenna system 610 may comprise one or more antennae and one or more multiplexors (not shown) that perform a switching function to provide the antenna system 610 with transmit and receive signal paths. In the receive path, received RF signals can be coupled from a multiplexor to a low noise amplifier (not shown) that amplifies the received RF signal and sends the amplified signal to the radio system 615.

In alternative embodiments, the radio system 615 may comprise one or more radios that are configured to communicate over various frequencies. In one embodiment, the radio system 615 may combine a demodulator (not shown) and modulator (not shown) in one integrated circuit (“IC”). The demodulator and modulator can also be separate components. In the incoming path, the demodulator strips away the RF carrier signal leaving a baseband receive audio signal, which is sent from the radio system 615 to the baseband system 620.

If the received signal contains audio information, then baseband system 620 decodes the signal and converts it to an analog signal. Then the signal is amplified and sent to a speaker. The baseband system 620 also receives analog audio signals from a microphone. These analog audio signals are converted to digital signals and encoded by the baseband system 620. The baseband system 620 also codes the digital signals for transmission and generates a baseband transmit audio signal that is routed to the modulator portion of the radio system 615. The modulator mixes the baseband transmit audio signal with an RF carrier signal generating an RF transmit signal that is routed to the antenna system and may pass through a power amplifier (not shown). The power amplifier amplifies the RF transmit signal and routes it to the antenna system 610 where the signal is switched to the antenna port for transmission.

The baseband system 620 is also communicatively coupled with the processor 560. The central processing unit 560 has access to data storage areas 565 and 570. The central processing unit 560 is preferably configured to execute instructions (i.e., computer programs or software) that can be stored in the memory 565 or the secondary memory 570. Computer programs can also be received from the baseband processor 610 and stored in the data storage area 565 or in secondary memory 570, or executed upon receipt. Such computer programs, when executed, enable the system 550 to perform the various functions of the present invention as previously described. For example, data storage areas 565 may include various software modules (not shown) that are executable by processor 560.

Various embodiments may also be implemented primarily in hardware using, for example, components such as application specific integrated circuits (“ASICs”), or field programmable gate arrays (“FPGAs”). Implementation of a hardware state machine capable of performing the functions described herein will also be apparent to those skilled in the relevant art. Various embodiments may also be implemented using a combination of both hardware and software.

Furthermore, those of skill in the art will appreciate that the various illustrative logical blocks, modules, circuits, and method steps described in connection with the above described figures and the embodiments disclosed herein can often be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module, block, circuit or step is for ease of description. Specific functions or steps can be moved from one module, block or circuit to another without departing from the invention.

Moreover, the various illustrative logical blocks, modules, and methods described in connection with the embodiments disclosed herein can be implemented or performed with a general purpose processor, a digital signal processor (“DSP”), an ASIC, FPGA or other programmable logic device, discrete gate or transistor logic, discrete hardware components, or any combination thereof designed to perform the functions described herein. A general-purpose processor can be a microprocessor, but in the alternative, the processor can be any processor, controller, microcontroller, or state machine. A processor can also be implemented as a combination of computing devices, for example, a combination of a DSP and a microprocessor, a plurality of microprocessors, one or more microprocessors in conjunction with a DSP core, or any other such configuration.

Additionally, the steps of a method or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such the processor can read information from, and write information to, the storage medium. In the alternative, the storage medium can be integral to the processor. The processor and the storage medium can also reside in an ASIC.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

1. A fetal and maternal monitoring system, the system comprising:
one or more sensing devices in contact with a user for detecting signals;
a central control device in wired communication with the one or more sensing devices, wherein the central control device is configured to provide power to the one or more sensing devices, and wherein the central control device is configured to receive and process the detected signals into signal data; and
a gateway device in wireless communication with the central control device to receive the signal data and display it to a user on a display.

2. The system of claim 1, further comprising a remote server in communication with the gateway device over a network to receive and store the signal data.

3. The system of claim 1, further comprising a remote user device in communication with the remote server to display the signal data to a remote user.

4. A method for fetal and maternal monitoring, comprising:
detecting signals from a human body using one or more sensing devices;
receiving and processing the detected signals at a central control device to produce signal data, wherein the central processing device is configured to provide power to the one or more sensing devices;
receiving the signal data at a gateway device for display to a user;
transmitting the signal data to a remote user device over a network; and
receiving feedback from the remote user device and displaying the feedback on the gateway device.

5. A method for performing an interactive remote medical examination, comprising:
displaying instructions to a user on a gateway device for positioning one or more sensing devices in contact with the user to detect signals from the user;
transmitting the detected signals from the gateway device to a remote server for review by a medical professional; and
transmitting feedback generated by the medical professional at the remote server to the gateway device for display to the user.

6. The method of claim 5, wherein the instructions further comprise instructions for adjusting the one or more sensing devices to improve a quality of the detected signals.

7. The method of claim 5, wherein the gateway device and remote server are configured for real-time communication between the user and the medical professional.

8. The method of claim 5, further comprising prompting the user to answer at least one health-related question and transmitting an answer to the at least one health-related question to the remote server.

9. A sensing device with a visual feedback indicator, comprising:
a sensing device in contact with a user for detecting signals; and
at least one visual indicator positioned on an external surface of the sensing device to indicate a status of the sensing device.

10. The device of claim 9, wherein the status of the sensing device indicates whether a signal is being detected.

11. The device of claim 10, wherein the status of the sensing device indicates a strength of a detected signal.

12. The device of claim 11, wherein the visual indicator is a light which changes color to indicate the status of the sensing device

13. The device of claim 11, wherein the visual indicator is a light which flashes in at least one pattern to indicate the status of the sensing device.
